(19) United States
(12) Patent Application Publication
(10) Pub. No.: US 2025/0339146 A1
(43) Pub. Date: Nov. 6, 2025

(54) Needle Driver

(71) Applicants: Dennis G. Higginbotham, Pittsburg, KS (US); Ryan I. Higginbotham, Pittsburg, KS (US); Simon J. Higginbotham, Pittsburg, KS (US); Aaron M. Higginbotham, Pittsburg, KS (US)

(72) Inventors: Dennis G. Higginbotham, Pittsburg, KS (US); Ryan I. Higginbotham, Pittsburg, KS (US); Simon J. Higginbotham, Pittsburg, KS (US); Aaron M. Higginbotham, Pittsburg, KS (US)

(21) Appl. No.: 19/172,698

(22) Filed: Apr. 8, 2025

Related U.S. Application Data
(60) Provisional application No. 63/642,355, filed on May 3, 2024.

(51) Int. Cl. A61B 17/062; A61B 17/00

(52) U.S. Cl. CPC A61B 17/062; A61B 2017/00407; A61B 2017/00424

(57) ABSTRACT

A needle driver is provided. The needle driver includes a pair of elongated members each having a proximal end and a distal end, wherein the elongated members are connected by a pin pivot to allow scissor-like movement between open and closed positions. A first jaw and a second jaw are located at the distal ends of the elongated members and are able to grip a surgical needle. Each elongated member includes a handle at the proximal end with a finger hole and a palm member positioned between the handle and the pin pivot. The palm members include flat interior faces that extend laterally beyond the edges of the elongated members to distribute pressure across the user's palm. A ratchet mechanism positioned between the handle and palm member selectively locks and unlocks the jaws. The needle driver improves ergonomic comfort, increases surgical precision, and supports ambidextrous operation during suturing procedures.

### CROSS REFERENCE TO RELATED APPLICATION

This application claims the benefit of pending U.S. provisional application No. 63/642,355 filed on May 3, 2024. The above identified patent application is herein incorporated by reference in its entirety.

### BACKGROUND OF THE INVENTION

The present invention relates to surgical instruments, more specifically, to needle drivers used in suturing procedures. Needle drivers are a fundamental component of surgical toolkits, enabling surgeons to grasp and guide suturing needles with precision. These instruments typically resemble scissors and consist of a pair of pivotally connected elongated members terminating in jaws that hold the needle, with finger rings at the opposite end to allow for manual operation. Despite their widespread use and familiarity among medical professionals, conventional needle driver designs pose several ergonomic and functional challenges that can negatively impact surgical performance, particularly during prolonged or delicate procedures.

Current needle driver designs generally require the user to insert their thumb and a finger into opposing rings and apply a scissor-like motion to manipulate the jaws. This ring-based design creates several drawbacks. First, the restricted range of motion caused by the rigid finger rings often limits wrist and hand mobility, which can reduce fine motor control and dexterity. Second, the confined grip configuration required by the finger rings can lead to localized pressure points and discomfort, especially during lengthy procedures. This physical strain increases fatigue and can affect a surgeon's precision and efficiency. Additionally, the need to maintain a firm grip within the finger rings restricts the user from freely adjusting hand position or switching grip techniques during use.

Although some modern needle driver variants incorporate locking mechanisms to maintain grip on the needle without continuous pressure, these mechanisms often necessitate awkward thumb manipulation to engage or disengage the jaws. This further limits the ability to rapidly transition between needle grasping and repositioning, which is particularly critical in complex or minimally invasive surgical environments. Moreover, existing designs offer limited accommodation for the “palming” technique, a preferred method by many experienced surgeons wherein the instrument is cradled in the palm to increase control and tactile feedback. Standard needle drivers lack sufficient surface area or ergonomic shaping along the body of the instrument to support this technique effectively.

Some attempts have been made to improve the ergonomics of needle drivers by modifying handle shapes or introducing soft-grip materials. However, these modifications often do not extend to the critical interface between the tool and the palm or fail to allow seamless transition between finger-ring control and palming. Additionally, few designs offer true ambidextrous operation, typically requiring the device to be specially manufactured for either right-handed or left-handed use. This limits their utility in diverse surgical environments and can necessitate duplicative inventory.

Therefore, there remains a need for a needle driver that overcomes the ergonomic constraints and functional limitations of existing models. Specifically, there is a need for an instrument that enables intuitive and precise needle control without requiring constant finger-ring engagement, provides improved comfort through even pressure distribution across the palm, and supports both right- and left-handed users with equal effectiveness. The present invention addresses these unmet needs by incorporating novel palm support features, an integrated ratchet mechanism for effortless locking, and a design to facilitate ambidextrous use, thereby offering a more versatile and user-friendly solution for modern surgical suturing.

In light of the devices disclosed in the known art, it is submitted that the present invention substantially diverges in design elements and methods from the known art and consequently it is clear that there is a need in the art for an improvement for a needle driver. In this regard the instant invention substantially fulfills these needs.

### SUMMARY OF THE INVENTION

In view of the foregoing disadvantages inherent in the known types of needle drivers now present in the known art, the present invention provides a new needle driver wherein the same can be utilized for improving user comfort and enhancing functionality during surgical suturing.

It is an objective of the present invention to provide a needle driver comprising a pair of elongated members connected by a pin pivot to allow scissor-like articulation for opening and closing the jaws. The elongated members terminate at their distal ends in a first jaw and a second jaw, which are configured to securely grip a surgical needle during suturing procedures. This configuration enables a controlled and repeatable grasp of the needle, facilitating accurate placement of sutures in both superficial and deep tissue layers.

It is an objective of the present invention to provide a needle driver that includes a palm member positioned between each handle and the pin pivot, wherein the palm members comprise a flat face on an interior side of each elongated member and extend laterally beyond both the interior and exterior edges. The palm members are configured to distribute pressure across the palm of the user during operation. This structure improves ergonomic performance by reducing localized pressure and operator fatigue during extended surgical procedures.

It is an objective of the present invention to provide a needle driver having a ratchet mechanism positioned between each handle and its respective palm member. The ratchet mechanism is configured to selectively lock and unlock the jaws in one or more positions, enabling the user to maintain needle grip without continuous pressure. This mechanical configuration allows for temporary fixation of the needle, providing for increased stability and minimizing the need for repositioning during suturing.

It is an objective of the present invention to provide a needle driver configured for ambidextrous operation by incorporating structural substantially symmetry along both elongated members. As a result, the needle driver fully supports ambidextrous operation, allowing both right-handed and left-handed users to utilize the instrument with equal precision and efficiency.

It is an objective of the present invention to provide a needle driver wherein each handle includes a finger hole dimensioned to receive the user's finger and provide control of the instrument during operation. The finger holes are shaped to accommodate a range of finger sizes while maintaining consistent tactile feedback.

It is an objective of the present invention to provide a needle driver formed from surgical-grade stainless steel to ensure biocompatibility, corrosion resistance, and suitability for repeated sterilization. The material ensures that the device maintains structural integrity under surgical conditions, as well as compliance with operating room hygiene standards.

It is an objective of the present invention to provide a needle driver with jaws that include textured inner surfaces to improve grip on surgical needles. The textured surface increases friction between the jaws and the needle, reducing the likelihood of needle slippage.

It is an objective of the present invention to provide a needle driver wherein the palm members are shaped and positioned to enable the instrument to be operated using a palming technique. This technique involves resting the tool in the palm rather than relying solely on finger ring actuation. The design supports greater dexterity and finer control during delicate suturing procedures.

It is therefore an object of the present invention to provide a new and improved needle driver comprising that has all of the advantages of the known art and none of the disadvantages.

Other objects, features, and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings.

FIG. 1 shows a perspective view of an embodiment of the needle driver in a closed position.

FIG. 2 shows a perspective view of an embodiment of the needle driver in an open position.

FIG. 3 shows a top elevation view of an embodiment of the needle driver.

FIG. 4 shows a side view of a first elongated member of an embodiment of the needle driver.

FIG. 5 shows a perspective view of a first elongated member of an embodiment of the needle driver.

FIG. 6 shows a perspective view of a second elongated member of an embodiment of the needle driver.

### DETAILED DESCRIPTION OF THE INVENTION

Reference is made herein to the attached drawings. For the purpose of presenting a brief and clear description of the present invention, the preferred embodiment will be discussed as being used for providing a needle driver designed to enhance user comfort and functionality during surgical suturing procedures. The figures are intended for representative purposes only and should not be considered to be limiting in any respect.

Reference will now be made in detail to the exemplary embodiment(s) of the invention. References to “one embodiment,” “at least one embodiment,” “an embodiment,” “one example,” “an example,” “for example,” and so on indicate that the embodiment(s) or example(s) may include a feature, structure, characteristic, property, element, or limitation but that not every embodiment or example necessarily includes that feature, structure, characteristic, property, element, or limitation. Further, repeated use of the phrase “in an embodiment”, “first embodiment”, “second embodiment”, or “third embodiment” does not necessarily refer to the same embodiment.

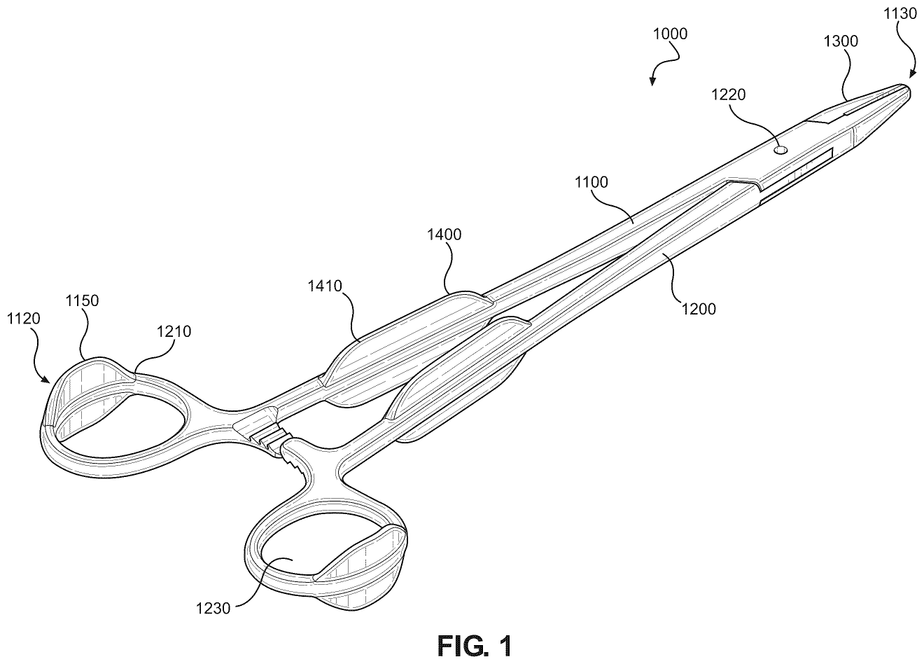

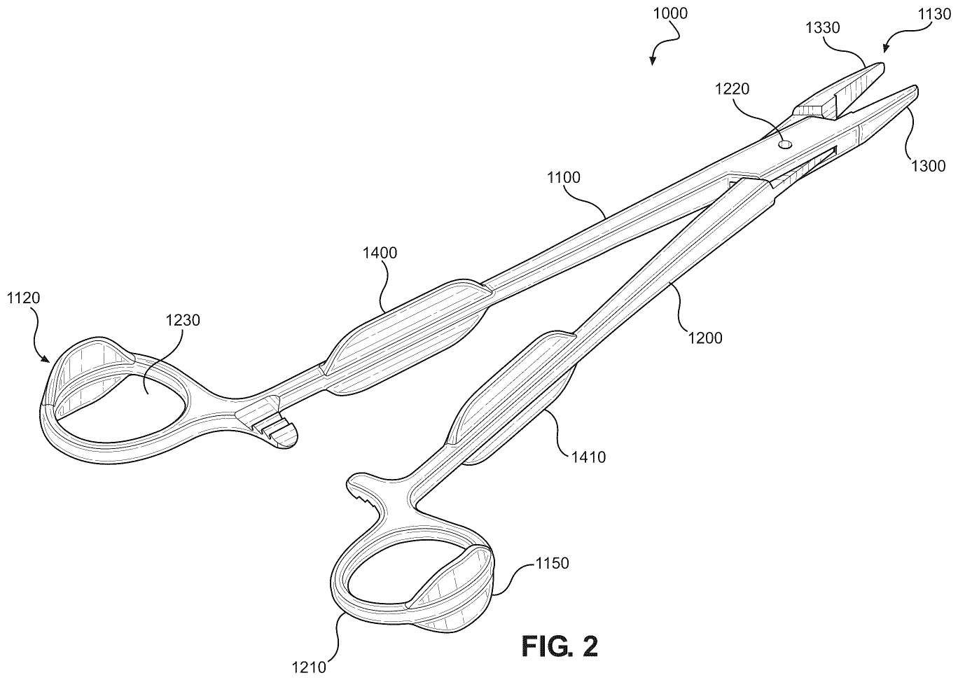

Referring now to FIGS. 1 and 2, there is shown a perspective view of an embodiment of the needle driver in a closed position and in an open position, respectively. In the illustrated embodiment, the needle driver 1000 includes a pair of elongated members 1100, 1200 each having a proximal end 1120 and a distal end 1130, wherein the proximal end 1120 is configured to form a handle portion 1210 and the distal end 1130 terminates in a jaw assembly 1300. The elongated members 1100, 1200 are pivotally coupled at a central midpoint via a pin pivot 1220, which defines an axis of rotation and enables angular displacement of the members 1100, 1200 in a scissor-like motion. This pivotal connection allows the distal ends 1130 to transition between an open configuration, wherein the jaws 1300 are spaced apart, and a closed configuration, wherein the jaws 1300 converge.

The distal ends 1130 of the elongated members 1100, 1200 are integrally formed with a first jaw and a second jaw, respectively, which together constitute the jaw assembly 1300 adapted to grip a surgical needle. In the closed configuration, the inner surfaces of the jaws 1300 are positioned in aligned opposition, forming a compressive interface configured to securely hold the body of a surgical needle for manipulation during suturing procedures. In some embodiments, the jaws 1300 include textured or serrated gripping surfaces 1330 to provide increased frictional engagement with the needle and reduce slippage during use.

Each elongated member 1100, 1200 includes the handle portion 1210 disposed at the proximal end 1120, configured to facilitate manual manipulation and control of the needle driver 1000 during surgical procedures. In the illustrated embodiment, each handle 1210 comprises an annular finger hole 1230 defined by a rigid peripheral wall, configured to receive the thumb and finger of a user, such as a surgeon, and permit rotational and compressive actuation of the elongated members 1100, 1200 about the pivot axis. The finger holes 1230 are sized to accommodate a range of finger diameters, and their geometry may include circular, elliptical, or contoured profiles to optimize ergonomic comfort, tactile feedback, and instrument stability during use.

In the illustrated embodiment, the needle driver 1000 includes lateral flanges 1150 disposed around the exterior of each finger hole 1230 located at the proximal ends of the elongated members 1100, 1200. The lateral flanges 1150 extend radially outward from the outer circumference of the finger holes 1230 and are either integrally formed with or affixed to the handle portion 1210 of each elongated member 1100, 1200. Each flange 1150 comprises a generally planar or contoured projection configured to increase the surface area in contact with the user's fingers during manipulation of the instrument. The lateral flanges 1150 function to enhance grip stability, distribute contact pressure more evenly, and minimize localized strain on the user's fingers during prolonged use.

In some embodiments, the flanges 1150 include ergonomic contouring, chamfered or rounded edges, and surface textures or patterns to improve tactile feedback and reduce slippage. The lateral extension of these flanges beyond the primary handle body also serves as a stabilizing structure, allowing the user to exert greater control over the angular orientation of the needle driver 1000 during precision suturing. This configuration is beneficial when alternating between traditional finger insertion and palming grip techniques, as the flanges provide additional leverage and resistance against unintentional rotation or misalignment.

A palm member 1400 is integrally or operably positioned between the handle 1210 and the pin pivot 1220 along the longitudinal axis of each elongated member 1100, 1200, forming an intermediate engagement interface between the user's palm and the body of the instrument. In the illustrated embodiment, each palm member 1400 includes a substantially planar engagement surface oriented along the interior-facing side of the elongated member 1100, with lateral projections 1410 that project outward beyond both the interior and exterior edges of the elongated member 1100. The lateral projections 1410 increase the surface area available for palm contact, thereby enhancing mechanical leverage and distributing compressive forces more uniformly across the user's palm during manipulation of the needle driver 1000. The palm engagement surface comprises a flat or gently contoured geometry and may incorporate surface texturing, radiused transitions, or elastomeric overlays to improve tactile comfort, reduce localized pressure, and promote extended instrument use without inducing fatigue. The palm members 1400 are dimensionally and geometrically symmetrical relative to one another, enabling consistent performance regardless of whether the instrument is operated with the left or right hand. The position of each palm member 1400 are disposed along the arm of each elongated member 1100, 1200 to provide a biomechanical fulcrum point that enhances control and stability during precise suturing maneuvers.

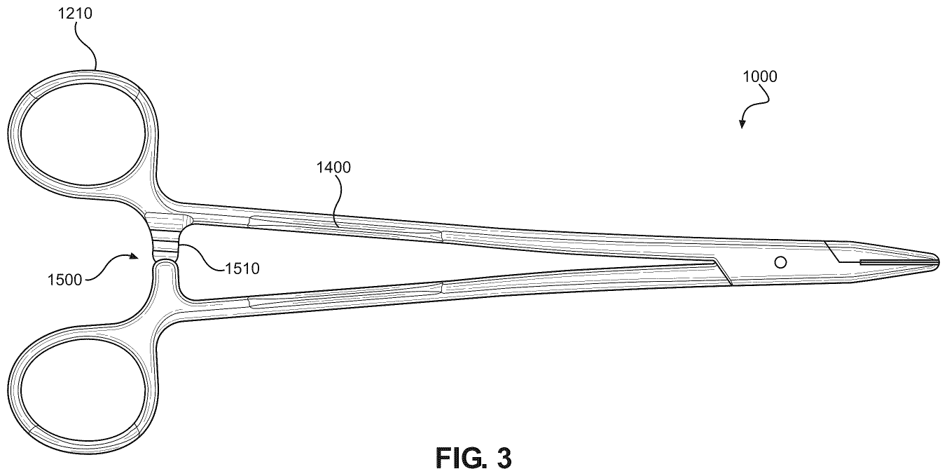

Referring now to FIG. 3, there is shown a top elevation view of an embodiment of the needle driver. In the illustrated embodiment, a ratchet mechanism 1500 is operatively positioned between the handle 1210 and the palm member 1400 of at least one elongated member 1100, configured to enable selective locking and unlocking of the jaw assembly 1300. The ratchet mechanism 1500 comprises an interlocking engagement system that includes a which can be actuated manually by the user's fingers during operation. In the shown embodiment, the interlocking engagement system comprises a pair of opposing toothed racks 1510, each integrally formed on the interior surface of the respective elongated members 1100, 1200 between the handle 1210 and the palm member 1400. The toothed racks 1510 extend from an interior side of the elongated members and are oriented to face one another such that the teeth of one rack are aligned with the teeth of the opposing rack when the elongated members 1100, 1200 are brought together. Each tooth is configured with an angular or sawtooth profile to allow for controlled engagement under applied compressive force. When the needle driver 1000 is actuated into the closed configuration, a tooth on one elongated member interlocks with a corresponding tooth on the opposing member, thereby securing the jaws 1300 in a locked position. The engagement of the interlocking teeth prevents unintended separation of the jaws and maintains a consistent clamping force on the surgical needle. In the illustrated embodiment, toothed rack is separated by applying sufficient manual force to overcome the mechanical resistance between the teeth, allowing the user to unlock and open the jaws as needed. The toothed rack 1510 allows for a tool-less locking functionality without requiring rotation or thumb manipulation, providing for uninterrupted suturing operations.

In alternate embodiments, the needle driver comprises any suitable locking system configured to maintain the jaws in a closed position during use. Such systems may include, but are not limited to, single-sided ratchets, cam-based locking mechanisms, spring-biased latches, or magnetic engagement features. The selected locking mechanism may vary depending on the intended surgical application, desired tactile feedback, or manufacturing requirements, provided it enables reliable, repeatable, and user-controlled engagement and disengagement of the jaws.

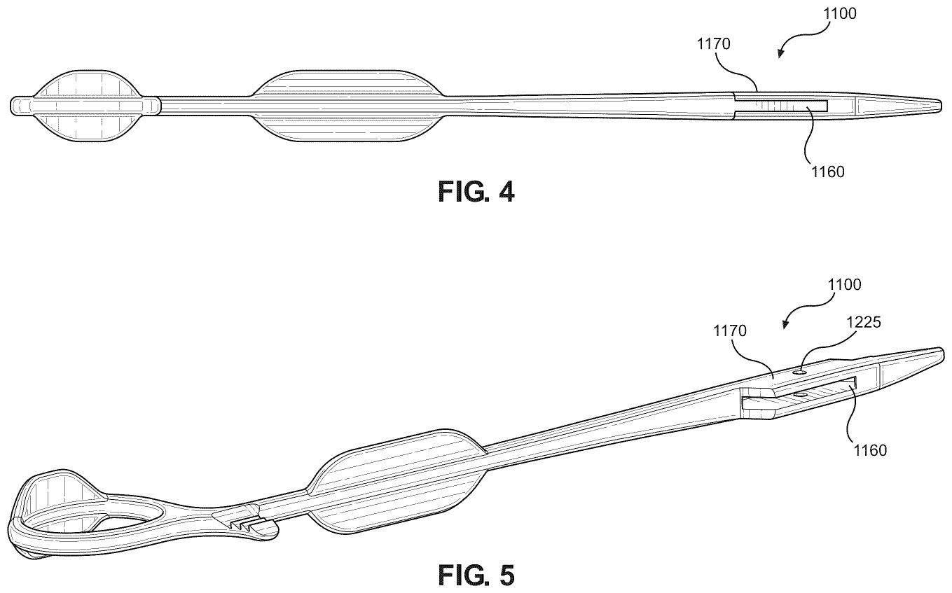

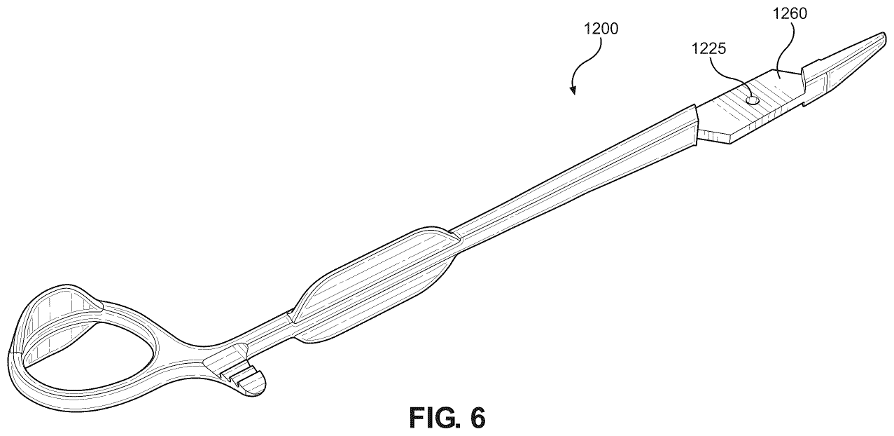

Referring now to FIGS. 4-6, there is shown a side view of a first elongated member of an embodiment of the needle driver, a perspective view of a first elongated member of an embodiment of the needle driver, and a perspective view of a second elongated member of an embodiment of the needle driver, respectively. In the illustrated embodiment, the first elongated member 1100 includes a recessed interface region 1160 located near the distal end, configured to receive a complementary portion of the second elongated member 1200 to facilitate scissor-like articulation. The interface region 1160 comprises a slot or channel formed transversely through the body of the first elongated member 1100, defined by opposing lateral walls 1170 and an interior surface. The slot is dimensioned to accommodate a flattened segment 1260 of the second elongated member 1200, enabling overlapping engagement between the two members. Aligned through-holes 1225 extend transversely across the opposing lateral walls 1170 and the flattened segment 1260, allowing for insertion of a pivot pin that passes through both the first and second elongated members. This construction ensures secure coupling of the members while permitting rotational movement about the pivot axis. The recessed design of the first elongated member enables the assembled configuration to remain compact and planar, minimizing the overall profile of the needle driver and facilitating smooth actuation during use.

In some embodiments, the elongated members 1100 and 1200 are configured with substantially similar geometries and ergonomic features on either side of a central vertical plane bisecting the pin pivot. While structural asymmetry exists at the pivot interface—where elongated member 1100 includes a recessed slot and elongated member 1200 includes a corresponding flat interface portion—the external contours, palm members, handles, and jaws are dimensioned and aligned to maintain overall functional and visual balance. This configuration enables ambidextrous operation of the needle driver 1000, allowing both right-handed and left-handed users to manipulate the instrument with equal efficiency and without requiring mechanical adjustment or reconfiguration.

The needle driver is configured to enhance ergonomic performance and operational functionality during surgical suturing procedures, thereby improving precision, reducing user fatigue, and increasing procedural efficiency. During operation, a surgeon inserts the thumb and finger into the respective finger holes of the proximal handles while positioning the palm against the planar engagement surface of the palm members, which serve to stabilize the instrument and distribute applied forces across a broader surface area. Once the surgical needle is positioned between the opposing jaws, the surgeon actuates the ratchet mechanism-typically via a finger-accessible lever-causing the locking pawl to engage the toothed rack and secure the jaws in the closed configuration, thereby clamping the needle with consistent compressive force. Throughout the suturing procedure, the surgeon may adjust the angular orientation and grip pressure of the needle driver as needed, aided by the balanced mass distribution and ergonomic contact points provided by the instrument's geometry. Upon completion of a suture pass, the ratchet mechanism is disengaged, allowing the jaws to return to the open configuration and release the needle. This configuration supports smooth transitions between suture placements while minimizing hand strain and enhancing procedural control. In the illustrated embodiment, the elongated members, including handles, palm members, and jaws, are constructed from surgical-grade stainless steel. This material choice ensures durability, corrosion resistance, and suitability for sterilization.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, device and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A needle driver, comprising:
a pair of elongated members each having a proximal end and a distal end, the elongated members connected at a midpoint by a pin pivot and configured to facilitate scissor-like movement between an open position and a closed position;
a first jaw disposed at the distal end of a first one of the elongated members, and a second jaw disposed at the distal end of a second one of the elongated members, the jaws configured to grip a surgical needle when the elongated members are in the closed position;
a handle disposed at the proximal end of each of the elongated members, each handle including a finger hole for receiving a user's finger;
a palm member disposed between the handle and the pin pivot on each of the elongated members, each palm member extending laterally beyond both an interior edge and an exterior edge of the respective elongated member, the palm members configured to engage a user's palm and distribute pressure during use;
a palm member positioned between the handle and the pin pivot on each elongated member, wherein the palm members include a flat face on an interior side extending laterally beyond the edges of the elongated members.

2. The needle driver of claim 1, wherein each of the palm members comprise a flat surface on an interior side of the elongated member.

3. The needle driver of claim 1, further comprising a ratchet mechanism operatively coupled between the elongated members and configured to selectively lock and unlock the jaws.

4. The needle driver of claim 3, wherein the ratchet mechanism is disposed between the handle and the palm member of each elongated member.

5. The needle driver of claim 3, wherein the ratchet mechanism comprises a lever accessible by a user's fingers for engaging and disengaging the jaws without requiring removal of fingers from the finger holes.

6. The needle driver of claim 1, wherein each of the elongated members, palm members, and jaws is formed from surgical-grade stainless steel.

7. The needle driver of claim 1, wherein each palm member has a uniform geometry configured to enable ambidextrous operation by both right-handed and left-handed users.

8. The needle driver of claim 1, wherein the elongated members, palm members, and jaws form a monolithic structure.

9. The needle driver of claim 1, wherein the proximal ends of the elongated members are symmetrical.

10. The needle driver of claim 1, wherein the jaws comprise textured inner surfaces configured to enhance needle retention.

11. The needle driver of claim 1, wherein the palm members are integrally formed with the elongated members as a single unitary structure.

12. The needle driver of claim 1, wherein the finger holes are shaped and sized to accommodate various finger diameters for improved ergonomic comfort.

13. The needle driver of claim 1, wherein the palm members are contoured to follow the curvature of a user's palm to enhance ergonomic comfort and reduce localized pressure during prolonged use.

14. The needle driver of claim 1, wherein the palm members include a textured or non-slip surface finish to improve grip and reduce slippage during surgical procedures.

15. The needle driver of claim 1, wherein the jaws are spring-biased toward the open position, requiring user-applied force to bring the jaws into the closed position.

16. The needle driver of claim 1, wherein the elongated members are connected at the midpoint by a low-friction pivot assembly comprising a bearing or bushing to ensure smooth and controlled scissor-like movement.

17. The needle driver of claim 1, wherein the palm members comprise a polymer or elastomeric material overmolded onto a metallic core.

18. The needle driver of claim 1, wherein the ratchet mechanism includes multiple detent positions to enable incremental locking of the jaws at varying levels of closure force.

19. The needle driver of claim 1, wherein the palm members are removably attached to the elongated members to allow for sterilization, replacement, or reconfiguration.

20. The needle driver of claim 1, wherein the palm members are angled relative to the longitudinal axis of the elongated members to promote a natural wrist posture during operation.
